(19) United States
(12) Patent Application Publication
(10) Pub. No.: US 2019/0031834 A1
(43) Pub. Date: Jan. 31, 2019

(54) AMPHIPHILIC COPOLYMERS THEIR PREPARATION AND USE FOR THE DELIVERY OF DRUGS

(71) Applicant: DISTRETTO TECNOLOGICO SICILIA MICRO E NANO SISTEMI S.C.A.R.L., Catania (IT)

(72) Inventors: Anna Rita BLANCO, Acireale (IT); Gennara CAVALLARO, Palermo (IT); Gaetano GIAMMONA, Palermo (IT); Mariano LICCIARDI, Palermo (IT); Glovanna PITARRESI, Palermo (IT); Domenico TROMBETTA, Rosali (IT)

(21) Appl. No.: 15/516,993

(22) PCT Filed: Oct. 9, 2014
(86) PCT No.: PCT/EP2014/071648

§ 371 Date: Apr. 5, 2017

(51) Int. Cl. C08G 81/00; C08G 69/48; C08G 63/91; C08G 69/44; C08G 65/08; A61K 9/00; A61K 9/107; A61K 9/51; A61K 47/34; A61K 31/44

(52) U.S. Cl. CPC C08G 81/00; C08G 69/48; C08G 63/912; C08G 69/44; A61K 31/44; A61K 9/0014; A61K 9/1075; A61K 9/5153; A61K 47/34; C08G 65/08

(57) ABSTRACT

New amphiphilic polymers of formula (I)

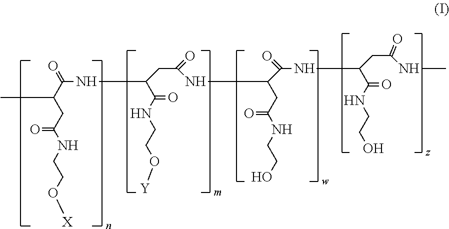

are described; the process for their preparation and their use as carriers for pharmaceutical drugs is also described.

### FIELD OF THE INVENTION

The present invention refers to new polymers and to their preparation and use as carriers for delivering pharmaceutical compounds

### STATE OF THE ART

One of the possible ways of delivering drugs having different physical, chemical and pharmacological properties is to use nano-scaled drug delivery systems (NSDDS), such as

nanoparticles, liposomes, dendrimers, or polymeric micelles.

Among NSDDS self-assembling nanoparticulate systems, have recently emerged as promising carriers for drug delivery and targeting since they are capable to maintain drug levels in the therapeutically desirable range and to increase drug solubility, stability, permeability and half-life.

These systems include polymeric micelles and polymeric nanoparticles and can be obtained by self-assembling of amphiphilic copolymers in which, in aqueous solution, hydrophilic and hydrophobic portions form a stable core-shell structure; they are capable of delivering a variety of drugs, including hydrophobic drugs whose clinical application is limited by their low solubility in aqueous solutions. They also improve delivery efficiency and reduce side effects by means of targeted delivery.

However, the development and synthesis of biocompatible amphiphilic copolymers able to self-assemble into micelles or nanoparticles useable as efficient carriers for delivering physically entrapped drug molecules is still in run and there is a constant need of more of these products in order to satisfy the necessities of the pharmaceutical industry.

### SUMMARY OF THE INVENTION

The invention refers to new amphiphilic polymers of formula (I) as described hereinafter and to a process for their preparation and their use.

### BRIEF DESCRIPTION OF THE FIGURES

FIG. 1 reports the cytocompatibility profiles of empty micelles on 16HBE cells after 4 h (a) and 24 h (b) of incubation by using different concentrations.

FIG. 2A and FIG. 2B show the activation of apoptatic cell death in NIH/3T3 mouse fibroblasts (A) and HUVECs (B) exposed for 24 h to NP suspensions. Caspase activation was determined by Western blot analysis of total cell extracts with specific antibodies against pro-caspase-3 (32 kDa) and its active form caspase-3 (17 kDa). Cultures not exposed were used as controls; camptothecin treated Jurkat lysate was used as positive control for apotosis. (PP: PHEA-Plga NPs; PPP: PHEA-Plga-Peg)

### DETAILED DESCRIPTION OF THE INVENTION

The present invention allows to overcome the above said problems making available polymers with polyaspartamide structure having formula (I)

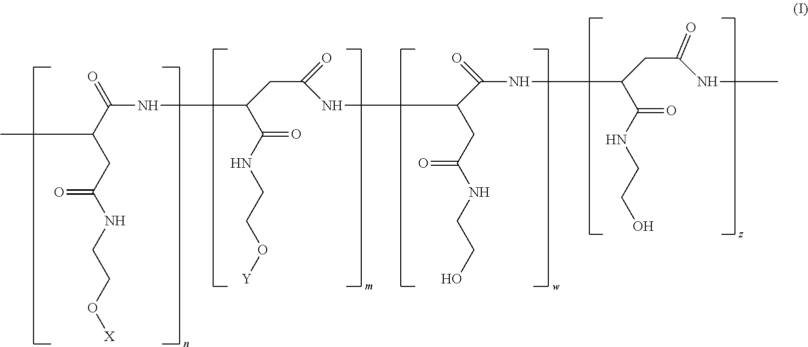

wherein

X— is chosen from the group consisting of H; —(C═O)—NH—CH₂—CH₂—(O—CH₂—CH₂)ₐ—OH or

—(C═O)—NH—CH₂—CH₂—(O—CH2—CH₂)ₐ—O—CH₃, where a is between 9 and 450;

Y— consisting of poly(lactic-co-glycolic) ester (PLEA) having a molecular weight between land 40 kDa;

where

n and m can be respectively between 0.1-50% of the total number of alpha and beta repeating units of the polymer, which are between 63 and 380;

w=total number of alpha and beta repeating units of the polymer—n; z=total number of alpha and beta repeating units of the polymer—m;

The X— groups in formula (I) are linked to the polymer PHEA, for example, by ester, urethane or carbonic linkages;

analogously when Y—, for example, is polylactide-glycolic chain it means that the carboxylic group of PLGA is linked to the polymer by ester linkage.

As it can be seen from the formula the present the polymers according to the invention present a biocompatible α,β-poly(N-2-hydroxyethyl)-d,l-aspartamide (PHEA) backbone and hydrophobic portions in the side chain consisting of polylactic-co-glycolic acid) (PLGA) chains.

PHEA is a synthetic water-soluble, biocompatible, nontoxic and nonantigenic polymer, which has been used for the preparation of colloidal drug-delivery systems, such as nanoparticles, micelles and to prepare polyelectrolytic complexes for gene delivery.

It is also known to modify the PHEA, in its the side chain, with hydrophilic chains, such as poly ethylene glycol (PEG), in combination with hydrophobic molecules in order to make it more biocompatible.

Usually, PEGs chains with molecular weight of 2000-5000 Da are used to form the hydrophilic outer shell of the polymeric micelles because provide important advantages including the micelles effective steric protection, prevent recognition by the reticuloendothelial system (RES) and prolong bloodstream circulation.

Preferred polymers of formula (I) are those where X— are directly conjugated to the polymer by urethane linkage; Y— are directly conjugated to the polymer by ester linkage.

More preferred are polymers of formula (I), wherein X— is H or —(C═O)—NH—CH2-CH2—(O—CH2-CH2)b-O—CH3, where a is 174; Y— consisting of polylactic-co-glycolic) ester (PLGA) having a molecular weight between 10-18 kDa.

The polymeric materials according to the invention can be used in the preparation of pharmaceutical compositions containing nano-scaled drug delivery systems, amphiphilic polyaspartamide graft-copolymers having: high biocompatibility, easy production method with high yields, reproducibility and low costs; versatility in terms of drug content and drug type, activity and administration route.

In fact the copolymers according to the invention are capable to self-assemble in water into micelles or nanoparticles type structure capable of loading (physically entrapping them) drug molecules belonging to the following therapeutic classes: steroid and non-steroid anti-inflammatory agents, antimicrobial agents such as aminoglycosides, macrolides, cephalosporin, tetracycline, quinolones, penicillin, beta-lactams, anti-glaucoma agents such as prostaglandins, prostamides, alpha- and beta-blockers, inhibitors of carbonic anhydrase, cannabinoids, antiviral agents, diagnostic agents, anti-angiogenic agents, antioxidants (among which for example silybin, sorafenib, desonide, curcumin); moreover the above said micelles or nanoparticles are capable to release the entrapped drugs in a prolonged and controlled time.

Therefore, the present invention refers also to pharmaceutical formulations where the copolymers object of the invention are used, the micelles can be prepared by water dispersion method or dialysis dispersion method. Nanoparticles can be prepared by homogenization-solvent evaporation method, water dispersion method, high pressure homogenization method.

The pharmaceutical formulations according to the description can be used either for topical or systemic administration for the treatment of various diseases for which find application all therapeutic classes above reported.

As an example, for the treatment and the prevention of all ocular diseases involving the posterior segment of the eye with neo-angiogenic and inflammatory component such as AMD (Age Macular Degeneration), diabetic retinopathies, macular edema, CNV (Choroideal Neo-Vascularization). Or always by way of example, for the treatment of disorders of the brain as glioma, Parkinson's disease, Alzheimer for which the classical therapeutic treatments are not effective. Typically therefore these pharmaceutical formulations may find application for the treatment of all those diseases for which the systems nano-scaled drug delivery systems (NSDDS) may offer therapeutic advantages.

Moreover the invention refers also to a process for the preparation of a polymer of formula (I) starting from a polymer of formula (II):

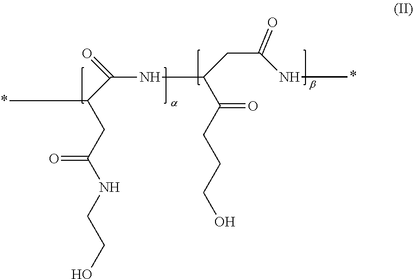

wherein:

α and β are the numbers of alpha and beta repeating units of the polymer, respectively, and are between 63 and 380

According to the invention the process of preparation of the polymer of formula (II) comprises the, following steps:

a) activation of the hydroxyl groups of the polymer (II) by a carbonylating agent;

b) reaction of the so activated compound with a PEG molecule bearing terminal amine group of formula NH₂—CH₂—CH₂—(O—CH₂—CH₂)ₐ—OH or NH₂—CH₂—CH₂—(O—CH₂—CH₂)ₐ—O—CH₃, where h is between 9 and 450

c) activation of the carboxylic groups of a PLGA having a molecular weight between 1 and 40 kDa by a carboxylic acid activating agent;

d) reaction of the activated compound of step (c) with polymer (II) to obtain compounds of formula (I) cul X═H and Y=PLGA,

or

d′) reaction of the activated compound of step (c) with the compound obtained in step (h) to obtain compounds of formula (I) wherein X is —(C═O)—NH—CH₂—CH₂—(O—CH₂—CH₂)ₐ—OH or

—(C═O)—NH—CH₂—CH₂—(O—CH₂—CH₂)ₐ—O—CH₃, where a is between 9 and 450 and Y═ is polylactic-co-glycol c;) ester (PLGA) having a molecular weight between 1 and 40 kDa.

Reactions (a), (b), (c), (c′) are preferably carried out in aprotic polar solvent, for example dimethyl formamide (DMF).

Carbonylating agent is preferably a phenyl-bis-carbonate, such as for example bis(4-nitrophenyl)carbonate (PNFC) or succimidyl-bis-carbonate, such as for example di-succinimidyl-carbonate (DCS).

Reaction (c) is carried out preferably in presence of appropriate carboxylic group activating agents (for example carbonyl-di-imidazole (CDI), 1-ethyl-3-(3-dimethylaminopropyl)-carbodiimide hydrochloride (EDC), Hydroxybenzotriazole (HOBT), N-hydroxy-succinimide (NHS).

For reactions (a), polymer activation degree can be varied by modulating concentration of hydroxyl group activating agents of formula phenyl-bis-carbonate, such as for example PNFC or succimidyl-bis-carbonate, such as for example DCS. For reactions (a), activation degree of hydroxyl groups of the polymer is depending on the molar ratio between starting polymer repeating units (R.U.) and mole ratios of activating agent (0.01-1), reaction time (1-24 h) and reaction temperature (−10-+60° C.). Thus, modulating these parameters it is possible to obtain an activation degree varying between 0.1 and 20 mol %.

The present invention will be better understood in view of the experimental examples.

Experimental Part

Synthesis of Polymer of Formula (I)

Polymer of formula (I) (PHEA-PEG-PLEA) was synthesized starting from the water soluble polymer poly(N-2-hydroxyethyl)-DL-aspartamide (PHEA) having average molecular weight (Mw) between 10 and 70 kDa (preferably 45 kDa) by two synthesis steps.

Hydroxyl groups present in the PHEA side chain were activated by reacting with disuccinimidyl-bis-carbonate (DSC), in DMF solution at 40° O. After activation reaction, PEG-NH₂ was added and mixture maintained at 25° C. for 18 h. Molar ratio between PHEA repeating units (Rt) and moles of activating agent, reaction time and moles of PEG-NH-₂ determine derivatization degree of polymer. For example, by using a RU/DSC moles ratio of 0.15, RU/PEG-NH₂ moles ratio of 0.15 and an activation time of 4 h it was obtained a derivatization degree in PEG of PHEA equal to 10 mol %. Reaction product was purified by exhaustive dialysis and lyophilized. PHEA-PEG was obtained with a yield of 85% respect starting PHEA.

Conjugation degree of PEG to RHEA was determined by ¹H-NMR spectroscopy.

Terminal carboxylic groups present in the PLGA chain were activated by reacting with carbonyldiimidazole (CDI), in DMF solution at 35° C. After activation reaction, activated PLGA was added in a PHEA-PEG DMF solution and mixture maintained at 35° C., for 18 h. Molar ratio between PHEA repeating units (RU) and moles of PLGA, reaction time and moles of activating agent determine derivatization degree of polymer. For example, by using a RU/PLGA moles ratio of 0.01, PLGA/CDI moles ratio of 1.2 and an activation time of 4 h it was obtained a derivatization degree in PLGA of DHEA PEG equal to 1 mol %. Reaction product was purified by exhaustive dialysis and lyophilized. PHEA-PEG-PLGA was obtained with a yield of 55% respect starting PHEA-PEG.

The sequence of the conjugation reactions and reagent ratios of the above described reactions can be modulated in function of the solubility of the final copolymer. For example, activated PLGA can be added in a PHEA DMF solution and then hydroxyl groups present in the PHEA side chain can be activated by reacting with disuccinimidyl-bis-carbonate (DSC), to reacting with PEG-NH₂. The final copolymer, in this case is named PHEA-PLGA-PEG.

Conjugation degree of PLGA to PHEA-PEG was determined by ¹H-NMR spectroscopy.

Average molecular weight (Mw) of PHEA-PEG-PLGA was determined by organic (DMF) SEC, and can be between 45,000 and 500,000 Da (preferably 95,000 Da), calculated by comparison with a calibration curve obtained by using PEG molecular weight standards ranging from 1 000 to 145000 Da.

¹H-NMR (DMF_{d7}): δ 8.49-8.05 (br, 2H, —CO—NH—), 5.49 (m, 1H, —CH—O—), 5.25 (m, 2H, —CH₂—O—) 4.70 (m, H, —NH—CH—), 4.2-4.O (—CH₂—O—CO—O—, —CH₂—O—CO—NH—), 3.63 (—O—CH₂—CH₂—O—), 3.50 (m, 2H, —NH—CH₂—), 3.31 (m, 2H, —CH₂—OH), 2.79 (m, 2H, —CH₂—CO—), 1.57 (m, 3H, —CH₃—).

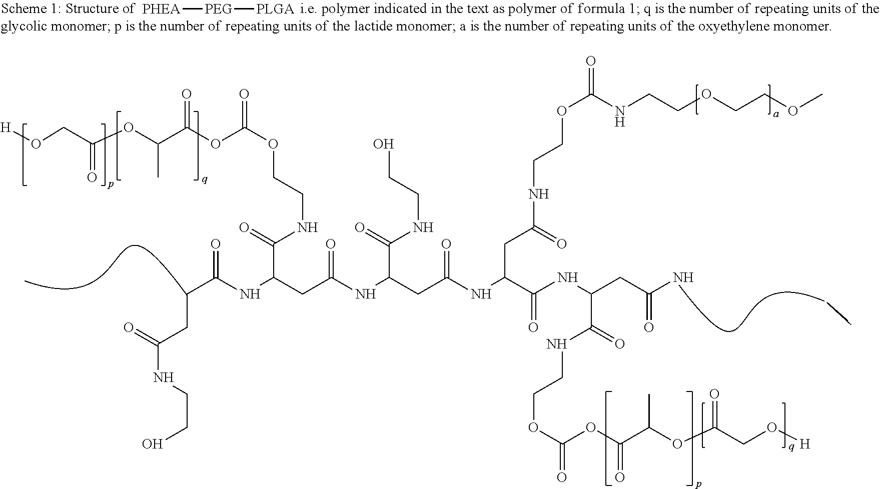

### EXAMPLE 1

### Synthesis of PHEA-PEG

To 400 mg of PHEA in a-DMF (5 mL), 97.21 mg of DSC were added and mixture stirred for 4 h at 40° C. After 4 h reaction amino-PEG₂₀₀₀ (740 mg) was added to this solution. After 18 h at 25° C., the PHEA.PEG was precipitated in diethyl ether (50 mL) and dialyzed against water through a membrane with nominal molecular weight cut off 3500. Yield 727.5 mg. The derivatization degree of PEG moiety (DDPEG %), calculate by ¹H NMR, was 9.6% with respect to the total amount of repeating units. ¹H-NMR (DMF_{d7}): 8.49-8.05 (br, 2H, —CO—NH—), 4.70 (m, H, —NH—CH—), 4.2 (—CH₂—O—CO—NH—), 3.63 (—C—CH₂—CH₂—O—), 3.50 (m, 2H, —NH—CH₂—), 3.31 (m, 2H, —CH₂—OH), 2.79 (m, 2H, —CH₂—CO—).

### EXAMPLE 2

### Synthesis of PHEA-PEG-PLGA

Obtained PHEA-PEG (400 mg, 1.14 mmol of repeating unit) was dispersed in DMF (8 mL). Separately, PLGA (polylactide-co-glycolic acid 50:50) (125 mg, 0.0114 mmol) was solubilized in DMF (8 mL) and CDI (1.85 mg, 0.0114 mmol) was then added at once. The reaction was maintained at 35° C. for 4 h under stirring. Then the activated PLGA was added to the PHEA-PEG solution dropwise. The reacting mixture was placed to react at 35° C. for 18 h. Afterwards, the PHEA-PRG-PLGA was precipitated in diethyl ether (150 mL) and the solid washed up with a mixture of diethyl ether/dichloromethane 2:1 (3×40 mL). Hence, the water soluble fraction was extracted with doubly distilled water (20 mL) and the solid product was recovered after freeze drying. Yield: 51%, The derivatization degree of PLGA moiety (DDPLGA %), calculate by ¹H NMR, was 1% with respect to the total amount of repeating units. ¹H-NMR (DMF_{d7}); 8.49-8.05 (br, 2H, —CO—NH—), 5.49 (m, 1H, —CH—O—), 5.25 (m, 2H, —CH₂—O—) 4.70 (m, H, —NH—CH—), 4.2-4.0 (—CH₂—O—CO—O—, —CH₂—O—CO—NH—), 3.63 (—O—CH₂—CH₂—O—), 3.50 (m, 2H, —NH—CH₂—), 3.31 (m, 2H, —CH₂—OH), 2.79 (m, 2H, —CH₂—CO—), 1.57 (m, 3H, —CH₃).

Average molecular weight (Mw) of PHEA-PEG-PLGA was determined by organic (DMF) SEC, and resulted 95,000 Da, calculated by comparison with a calibration curve obtained by using PEG molecular weight stardards ranging from 1000 to 145000 Da.

### EXAMPLE 3

### Determination of the Critical Aggregation Concentration (CAC) of the Polymers

The CAC of PHEA-PEG-PLGA, used as an example, was determined by fluorescence analysis, using pyrene as probe. A stock solution of pyrene (6.0×10-5M in acetone) was prepared and then aliquots of 20 μL were placed into vials and evaporated to remove acetone in an orbital shaker at 37° C., Subsequently, 2 mL of aqueous copolymer solution at concentrations ranging from 1×10−5 to 5 mg/mL were added to the pyrene residue; the final concentration of pyrene was 6.0×10−7M in each sample. The solutions were kept at 37° C. for 24 h under continuous stirring to equilibrate pyrene with micelles. Pyrene excitation and emission spectra were recorded at 37° C. using an emission wavelength of 373 nm and an excitation wavelength of 333 nm. Results are reported in Table 1.

TABLE 1
CAC of some prepared copolymers

| COPOLYMER | CAC (mg/ml) |
|---|---|
| PHEA-PEG-PLGA | 0.2 |

### EXAMPLE 4

### Preparation of Sorafenib Loaded Micelles from PHEA-PEG-PLGA

Typically, to a solution of polymer in DMF (2 mL, 20 mg/mL) sorafenib (10 mg) was added. The polymer/drug solution was then dried under vacuum (0.9 mbar) and, consequently, dispersed in PBS at pH 7.4 by means of sonication/vigorous mixing cycles (3×10 minutes). Afterwards the dispersion was placed into an orbital shaker for 18 h at 25° C., and so dialyzed against water though a membrane with nominal molecular weight cut off 1000. The resulting dispersion was then freeze dried and the product obtained as a yellow powder. The yields are reported in Table 2.

### EXAMPLE 5

### Determination of the Drug Payload of the DHEA-PEG-PLGA Based Nanocarriers

3 mg of drug loaded nanocarriers were dispersed in methanol/water 90:10 (5 mL) by sonicating for 10 minutes, and the dispersion was vigorous stirred for 4 h. After this time, it was filtered though a syringe filter of 0.2 μm and the methanol/water solution retrieved in an analytical flask (10 mL). The syringe filter was finally washed up with methanol/water until the solution was exactly diluted to 10 mL. 50 μL of this solution were analyzed by means of HPLC analysis: methanol/water (90:10) as eluant at flow rate of 1 mL/min and a C₆-phenyl column. Results are reported in Table 2.

### EXAMPLE 6

### Preparation of Sorafenib Loaded Nanoparticles from PHEA-PLGA-PEG

Preparation of nanoparticles from PHEA-PLGA-PEG: PHEA-PLEA-PEG (100 mg) was solubilized in THE/DMSO 50:50 (8 mL) and, then, polyvinylpyrrolidone (PVP, 80 mg) and soranefib (40 mg) were added at ones. The mixture was placed into a dialysis test tube with nominal molecular weight cut off 12-14 k and, consequently, dialyzed against TRIS buffer pH 7.5, 0.05M, for 4 hours. Finally, the nanoparticles were put into a dialysis tube with nominal molecular weight cut off 100 k and kept for 2 days against water.

The dispersion was filtered thought a 5 μm pore size syringe filter, freeze dried, obtaining solid nanoparticles. Yield: 100%

### EXAMPLE 7

### Determination of Size and Zeta Potential of the PHEA-PEG-PLGA Based Nanocarrier

The size distribution of the micelles was obtained by dynamic light scattering analysis performed on a Malvern Zetasizer NanoZS instrument at 25° C., fitted with a 532 nm laser at a fixed scattering angle of 173°. Aqueous solutions of micelles (2 mg/mL), were analysed after filtration through a 5 μm cellulose membrane filter. The intensity-average hydrodynamic diameter and polydispersity index (PDI) were obtained by cumulants analysis of the correlation function. The zeta potential (mV) was calculated from the electrophoretic mobility using the Smoluchowsky relationship and assuming that K a>>1 (where K and a are the Debye-Hückel parameter and particle radius, respectively). Results are reported in Table 2. As it can be seen, all copolymers shown ability to load the hydrophobic drug silybin.

TABLE 2
Hydrodynamic radius, polydispersity index (PDI), Zeta potential and drug
loading of the nanocarrier.

| | Average | | | |
|---|---|---|---|---|
| | Diameter | | Zeta Potential | Drug^{a} loading |
| Copolymer | (nm) | PDI | (mV) | (%) |
| PHEA-PEG- | 466 | 0.17 | −6.9 ± 3 | 8.77 ± 0.6 |
| PLGA | | | | |

^{a}Loaded drug refers to sorafenib.

### EXAMPLE 8

### In Vitro Cytocompatibility Evaluation

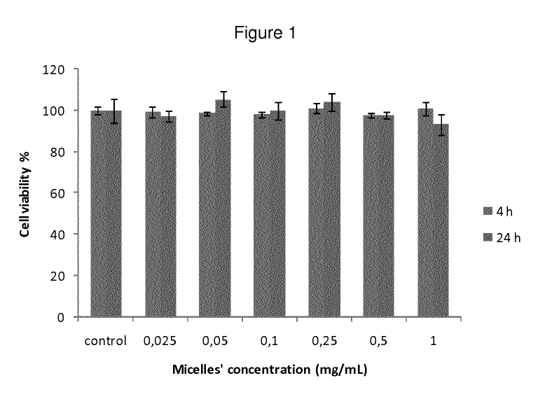

The biocompatibility of obtained micelles was assessed by the MTS assay on human bronchial epithelial (16HBE) cell line by using a commercially available kit (Cell Titer 96 Aqueous One Solution Cell Proliferation assay, Promega). Cells were seeded in 96 well plate at a density of 2×10⁴ cells/well and grown in Dulbecco's Minimum Essential Medium (DMEM) with 10% FBS (foetal bovine serum) and 1% of penicillin/streptomycin (10000 U/mL penicillin and 10 mg/mL streptomycin) at 37° C. in 5% CO2 humidified atmosphere. After 24 h of cell growth the medium was replaced with 200 μl of fresh culture medium containing unloaded PHEA-PEG-PLGA micelles at a concentration per well equal to 0.025, 0.05, 0.1, 0.25, 0.5 and 1 mg/ml. After 4 and 24 h of incubation time, DMEM was replaced with 100 μl of fresh medium, and 20 μl of a MTS solution was added to each well. Plates were incubated for an additional 2 h at 37° C. Cell incubated with medium was used as a negative control. Results were expressed as percentage reduction of the control cells (see FIG. 1). All culture experiments were performed in triplicates.

Results of cytocompatibility studies evidenced that all tested polymeric micelles, as empty micelles, are highly biocompatible and not toxic for normal human epithelial cells in vitro, thus constituting a potential efficient tool for delivering drugs in vivo.

### EXAMPLE 9

### Evaluation of Cytotoxicity of DHEA-PLEA and DHEA-Plga-Peg Nanoparticles

Cell culture and Treatments Two Cell Types were Used: NIH-1-3T3 Mouse Fibroblasts and Human

Umbilical Vein Endothelial Cells (HUVECs).

NIH/3T3 mouse fibroblasts (ATCC CRL-1658) were maintained in Dulbecco's Modified Eagle's Medium containing 10% fetal bovine serum and 100 U/mL penicillin-streptomycin at 37° C. in 5% CO2 with 95% humidity. The cells were plated into 24-well sterile plates (Nuns) at a concentration of 6.5×10⁴ cells per well and incubated in 500 μL of culture medium. After 24 hours, the culture medium was renewed, and the cells used for the experiments.

The HUVECs were isolated from freshly obtained human umbilical cords by collagenase digestion of the interior of the umbilical vein as described elsewhere (Jaffe et al., 1973), and were cultured in medium 199, supplemented with 20% of fetal bovine serum (FBS), 1% L-glutamine, 20 mM hepes, penicillin/streptomycin, 50 mg/ml endothelial cell growth factor, and 10 μg/mL heparin, in gelatin pretreated flasks. Cells were maintained in an incubator with humidified atmosphere containing 5% CO₂ at 37° C.

The nanoparticles (NPs) to be assayed were suspended in media by ultrasonication and added to cultures at concentrations ranging from 5.5 mg/ml to 0.075 mg/ml for 24 hours, after which cells were used to evaluate cell viability (by the sulforhodamine B assay) and apoptosis (by caspase-3 activation determination).

Sulforhodamine B (SRB) assay Without removing the cell culture supernatant, 125 μL of cold 50% (w/v) trichloroacetic acid (ICA) was added to each well (final TCA 10%), and the plates were incubated at 4° C. for 1 h. The plates were washed two times with water and then allowed to air-dry at room temperature. Three hundred μL of 4% (w/v) SRB solution in 1% (v/v) acetic acid were added to each well. Plates were left at room temperature for 30 min and then rinsed four times with 1% (v/v) acetic acid to remove unbound dye. The plates were allowed to air-dry at room temperature. The bound dye was extracted from the cells with a basic solution (Tris-base 10 mM) and the absorption of SRB was measured at 565 nm.

The intensity of the signal is proportional to the number of living cells and therefore a measure of their proliferation.

The LC₅₀, defined as the concentration of the product that kills 50% of cells, and 95% confidence limits were calculated according to Litchifield and Wilcoxon method (1949).

Western blot for caspase-3 evaluation Following appropriate treatment, cell lysates were prepared in non-denaturing buffer (10 mM Tris HCl, pH 7, 4, 150 mM NaCl, 1% Triton X-100, 5 mM EDTA-Na₂, 1 mM Dithiothreitol (DTT), 1 μg/mL leupeptin, 1 mM bent amidine, 2 μg/mL aprotinin). For immunoblot analyses, 40 μg of protein lysates per sample were denatured in 4× SDS-PAGE sample buffer [Tris-HCl 260 mM, pH 8.0, 40% (v/v) glycerol, 9.2% (w/v) SDS, 0.04% bromophenol bluem and 2-mercaptoethanol as reducing agent] and subjected to SDS-PAGE on 10% acrilamide/bisacrilamide gels. Separated proteins were transferred to nitrocellulose membrane (Hybond-P PVDF, Amersham Bioscience). Residual binding sites on the membrane were blocked by incubation in Tris Buffered Saline with Tween®20 (10 mM Tris, 100 mM NaCl, 0.1% Tween 20) and 5% (w/v) nonfat milk powder overnight at 4° C. Membranes were then probed with specific primary antibody rabbit anti-caspase-3 polyclonal antibody (Cell signaling Technology) followed by peroxidase-conjugated secondary antibody Ig (BD Pharmigen) (1:5000), and visualized with an ECL plus detection system (Amersham Biosciences). The equivalent loading of proteins in each well was confirmed by Ponceau staining and actin or Iamin B control.

Results: as shown in Tab. 1, PHEA-PLGA based nanocarriers possess a good biocompatibility on two cell lines (fibroblasts and cell endothelial cells), being HUVECs more resistant than fibroblasts. In fact, in all cases LC₅₀ values were >5.0 mg/ml, so leading to suppose that these nanocarriers might be useful to load a drug amount sufficient to induce a pharmacological response. Interestingly a good biocompatibility is evident also for PEGylated nanocarriers, that are generally more stable under physiological conditions.

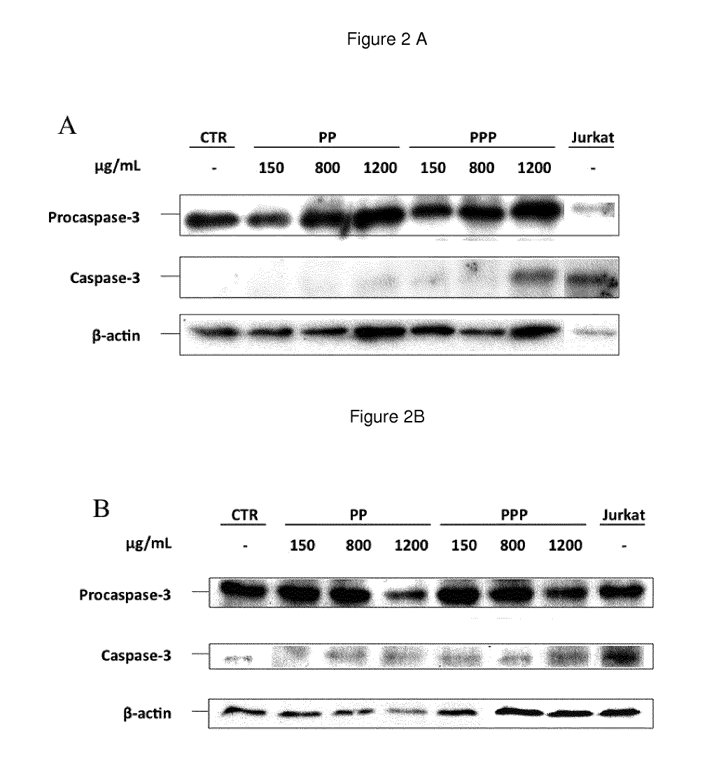

Furthermore, both on NIH/3T3 cells (FIG. 2 A) and HUVECs (FIG. 2B), until concentrations (1 mg/ml) able to induce only a light cytotoxic effect in the SRB assay (killing almost 2.0% of cells), these nanoccariers (PEGylated or not) do not activate the cell apoptotic machinery, so confirming their good biocompatibility.

TABLE 1
NPs cytotoxicity, as evaluated in the SRB assay, on NIH/3T3 and
HUVECs. LC₅₀s were calculated and 95% confidence intervals were
estimated. Data shown are a minimum of three independent
experiments done in quadruplicate.

| | | 95% confidence interval (mg/ml) | |
|---|---|---|---|
| NP treatment | LC₅₀ (mg/ml) | Lower | Upper |

NIH/3T3 cells

| | | | |
|---|---|---|---|
| PHEA-Plga | 5.6 | 4.9 | 6.2 |
| PHEA-Plga-Peg | 5.5 | 4.9 | 6.6 |

HUVECs

| | | | |
|---|---|---|---|
| PHEA-Plga | >15 | | |
| PHEA-Plga-Peg | 6.2 | 5.1 | 6.9 |

### EXAMPLE 10

### Evaluation of Capability of PHEA-PLGA and PHEA-Plga-Peg Nanoparticles to Cross Cell Barriers by In Vitro Transwell Assays

Cell Cultures

For epithelial barrier transport, Caco-2 cells were used. All experiments were performed from passages 25-30. The cells were seeded on polyethylene terephthalate membrane inserts (0.4 μm) fitted in bicameral chambers (Falcon™ Cell Culture Inserts, 10.5 mm ID, Corning Life Sciences DL, Corning, N.Y.) at 4.5×10⁴ cells/cm². The transepithelial electrical resistance (TEER) was tested by Millicell ERS meter (Fisher Sci., Pittsburgh, Pa.) to reflect the tightness of intercellular junctions and only cells with TEER≥250 Ωcm² were used for permeability study. At 18-20 d post seeding (90-100% confluence) on the trap swell, the cells were used for the experiments.

For endothelial permeability determinations, HUVECs were seeded on gelatin-coated polyethylene terephthalate membrane inserts (0.4 μm) (Falcon™ Cell Culture Inserts, 10.5 mm ID, Corning Life Sciences DL, Corning, N.Y.). The inserts were placed in 12-well culture plates, resulting in a two-compartment system separated by the membrane. Approximately 10⁵ HUVECs/cm² in 0.5 ml of complete medium were seeded at the upper side of the membrane, whereas 1.5 ml of complete medium was added to the lower compartment. These volumes prevented hydrostatic fluid pressures across the membranes. Both compartments were frequently replenished with complete medium as described. Cultures were grown for six days, resulting in the formation of confluent monolayers, which Was confirmed by phase contrast light microscopy.

Transport of NPs labelled with Fluorescein isothiocyanate (FITC). FITC-labelled NPs at non-cytotoxic concentrations (500 μg/ml), dissolved in complete media, were added to the apical chamber, then basolateral solutions were collected after 6, and 24 h. After 24 h, apical solutions were collected and membrane on the transwell insert was placed in 1.5 mL of ice-cold sodium hydroxide (0.5 M) and 1,5 sonicated with a probe-type sonic dismembrator. For FITC quantification, the apical and basolateral solutions were read spectrophotometrically (excitation 485 nm, emission 538 nm). Leakage of NP_{S}-FITC was defined by fluorescence in the bottom compartment and expressed as a percentage of total fluorescence (combined measurements in upper and lower compartments).

Assessment of endothelial layer functional integrity. Transendothelial albumin permeability was assessed as functional marker of endothelial layer integrity. HUVECs were cultured on Transwell inserts and exposed to non-cytotoxic concentration of NPs (500 μg/ml added to the upper compartment) for 24 h. The cells were then incubated with serum-free media for 1 h. Bovine serum albumin (BSA) (200 μM) was added to the apical chamber. Samples (50 μl) were taken from the basolateral chamber after 1 h and 2 h. The albumin content of the sample was determined with bromocresol green colorimetric assay kit (Sigma-Aldrich, Milano) using a calibration curve. Hydrogen peroxide was used in positive controls. Results: In research studies on NP transport across intestinal epithelial cells, Caco-2 cell line is generally accepted as the classical cell model owing to its homology and similar morphology with intestinal epithelial cells. In our experiments the quantitative measurement of FITC-labelled PHEA-Plga and PHEA-Plga-Peg NPs (Table 2) displayed that almost 5% of total NPs crossed cell monolayer to the bottom compartment of transwell plate after 24h, in accordance with the extremely high efficiency of this selectively permeable intestinal cell monolayer. When HUVECs were utilized to explore the transport of NPs through endothelium monolayer (Table 2), the results shoved an higher transport for both NPs, up to 26% and 33% of the total amount for PHEA-Plga and PHEA-Plga-Peg respectively.

TABLE 2
percentage of NPs transport in Caco-2 and HUVEC monolayers
(expressed as % of the concentration applied to the apical side of each
filter). Triplicate inserts were used in each experiment repetition.
Data are expressed as mean ± standard deviation.

| | |
|---|---|
| 6 hours | 24 hours |

Caco-2 transport (%)

| | | |
|---|---|---|
| PHEA-Plga | 0.9 ± 0.1 | 3.0 ± 0.1 |
| PHEA-Plga-Peg | 1.0 ± 0.2 | 4.2 ± 0.4 |

HUVECs transport (%)

| | | |
|---|---|---|
| PHEA-Plga | 4.6 ± 0.2 | 26.8 ± 0.9 |
| PHEA-Plga-Peg | 7.2 ± 0.4 | 33.2 ± 1.2 |

Transport of BSA across HUVEC confluent monolayers growing on 0.4-μm-pore transwell filters (from the top to the bottom chamber) was assessed in order to evaluate endothelial layer functional integrity. Both PHEA-Plga or PHEA-Plga-Peg NPs tested did not increase transport of albumin across HUVEC monolayers when compared to the negative control samples, so demonstrating that, even after incubation with PHEA-Plga or PHEA-Plga-Peg NPs for 24 h, the endothelial cell monolayer is still functionally intact.

1-14. (canceled)

15. A polymer of formula (I) 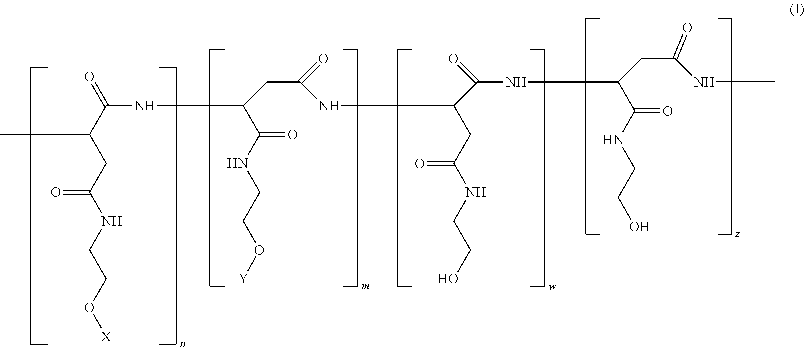
wherein;
X— is selected from the group consisting of H; —(C═O)—NH—CH₂—CH₂—(O—CH₂—CH₂)ₐ—OH or and
—(C═O)—NH—CH₂—CH₂—(O—CH₂—CH2)—O—CH₃, where a is between 9 and 450;
Y— is a poly(lactic-co-glycolic) ester (PLGA) having a molecular weight between 1 and 40 kDa;
n and m are, respectively, between 0.1-50% of the total number of alpha and beta repeating units of the polymer, which are between 63 and 380;
w=total number of alpha and beta repeating units of the polymer—n; and z=total number of alpha and beta repeating units of the polymer—m.

16. Polymers of formula (I) according to claim 15 wherein X— is directly conjugated to the polymer by urethane linkage and Y— is directly conjugated to the polymer by ester linkage.

17. Polymers of formula (I), wherein X— is H or —(C═O)—NH—CH2-CH2-(O—CH2-CH2)a-O—CH3, where a is 174; Y— is poly(lactic-co-glycolic) ester (PLGA) having a molecular weight between 10-18 kDa.

18. Pharmaceutical compositions containing polymers according to claim 15 as carriers for pharmaceutically active drugs.

19. Pharmaceutical compositions according to claim 18 wherein said pharmaceutically active drugs are chosen among drug molecules belonging to the following therapeutic classes: steroid and non-steroid anti-inflammatory agents, antimicrobial agents such as aminoglycosides, macrolides, cephalosporin, tetracycline, quinolones, penicillin, beta-lactams, anti-glaucoma agents such as prostaglandins, prostamides, alpha-and beta-blockers, inhibitors of carbonic anhydrase, cannabinoids, antiviral agents, diagnostic agents, anti-angiogenic agents, antioxidants.

20. Pharmaceutical compositions according to claim 18 wherein said polymers form micelle or nanoparticles loaded with an active principle.

21. Pharmaceutical compositions according to claim 18 in the form suitable for topical or systemic administration.

22. Pharmaceutical compositions according to claim 21 for use in the treatment of diseases for which nano-scaled drug delivery is suitable.

23. Pharmaceutical composition according to claim 22 wherein said diseases are ocular diseases involving the posterior segment of the eye with neo-angiogenic and inflammatory component.

24. Pharmaceutical composition according to claim 22 wherein said diseases are disorders of the brain.

25. Process for the preparation of a polymer of formula (I) wherein a polymer of formula (II): 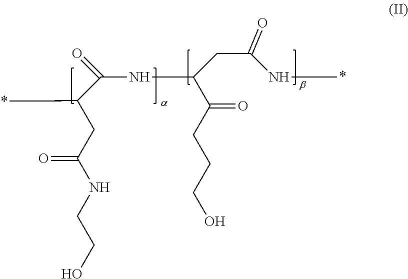
where:
α and β are the numbers of alpha and beta repeating units of the polymer, respectively, and are between 63 and 380
is submitted to the following reaction steps:
a) activation of the hydroxyl groups of the polymer (II) by a carbonylating agent;
b) reaction of the so activated compound with a PEG molecule bearing terminal amine group of formula NH₂—CH₂—CH₂—(O—CH₂—CH₂)ₐ—OH or NH₂—CH₂—CH₂—(O—CH₂—CH₂)ₐ—O—CH₃, where bis between 9 and 450;
c) activation of the carboxylic groups of a PLGA having a molecular weight between 1 and 40 kDa by a carboxylic add activating agent;
d) reaction of the activated compound of step (c) with polymer (II) to obtain compounds of formula (I) where X═H and Y=PLGA,
or
d′) reaction of the activated compound of step (c) with the compound obtained in step (b) to obtain compounds of formula (I) wherein X is —(C═O)—NH—CH₂—CH₂—(O—CH₂—CH₂)ₐ—OH or
—(C═O)—NH—CH₂—CH₂—(O—CH₂—CH₂)ₐ—O—CH₃, where a is between 9 and 450 and Y is poly(lactic-co-glycolic) ester (PLGA) having a molecular weight between 1 and 40 kDa.

26. Process according to claim 25 wherein steps (a), (b), (c) and (d) are carried out in aprotic polar solvent.

27. Process according to claim 25 wherein the carbonylating agent is a phenyl-bis-carbonate, or succimidyl-bis-carbonate.

28. Process according to claim 25 wherein step (c) is carded out in presence of: carbonyl-di-imidazole (CDI) or 1-ethyl-3-(3-dimethylaminopropyl)-carbodiimide hydrochloride (EDC) or Hydroxybenzotriazole (HOBT) or N-hydroxy-succinimide (NHS) as carboxylic group activating agents.
